(19) United States
(12) Patent Application Publication
(10) Pub. No.: US 2003/0083544 A1
(43) Pub. Date: May 1, 2003

(54) Method and apparatus for finding love

(76) Inventors: Catherine Richards, (US); W. Martin Snelgrove, (US)

Correspondence Address: W. MARTIN SNELGROUE, Suite 700, 312 ADELAIDE ST. W., Toronto, ON, M5V 1R2, CA

(21) Appl. No.: 10/279,731

(22) Filed: Oct. 25, 2002

Related U.S. Application Data
(60) Provisional application No. 60/330,566, filed on Oct. 25, 2001.

(51) Int. Cl. A61F 5/00

(52) U.S. Cl. 600/38

(57) ABSTRACT

An apparatus is presented which, carried by or embedded in a lonely or socially inept individual, communicates with like devices in such a way as to divine the likelihood of attraction due to relative sexual, social, intellectual or spiritual interests of the bearers. It may either be programmed explicitly by a trusted body, or suspect compatibility by observing and mining patterns of behaviour, environment and physiological response in the users of the said devices. The users are signalled or led to initial interaction in such a way as to maximize the likelihood of prolonged and deepened contact.

### PRIORITY CLAIM

The present application claims priority from U.S. provisional patent application No. 60/330,566, filed Oct. 25, 2001, the contents of which are incorporated here by reference.

### FIELD OF THE INVENTION

This invention relates to methods and apparatus for finding compatible individuals or groups, whether for romantic, sexual, intellectual, political or other liaisons.

### BACKGROUND OF THE INVENTION

People are lonely; they can mitigate this periodically in the company of others, but only some others are acceptable and they are becoming hard to find; partly because of dilution and partly because overt display of need can weaken negotiating positions and because deceit is prevalent. In general terms, they say they are searching for love.

Tastes are becoming increasingly specialized under the influence of modern marketing, making it more difficult for random interactions to bring acceptable pairs or groups together. Further, increased privacy obtained through such solo lifestyles and behaviours as living alone, watching television, surfing the net, playing videogames, telebanking, single-parenting, driving alone, telecommuting, and eating at drive-through restaurants reduces the number of random interactions that may lead to love. Love is also increasingly transient, again as marketing leads to the incessant manufacture of new desires which render obsolete any current relationship, so that there is an increased and relentless demand for finding partners.

### PRIOR ART

Matchmaking is an ancient art, but in modern cultures the conditions for it are no longer in place: stable communities and straightforward models of love and behaviour. All these contribute to keeping the size and complexity of the database of candidates small enough for manual processes. There is a need for a technological solution capable of finding love for people of very diverse characters with diverse and rapidly changing desires and emotional needs, and on a global scale.

Marcel Duchamp's Large Glass (1913) proposed an apparatus for transmission of desire based on his contemporary wireless transmission technologies. These technologies were those of spark telegraphy, Crookes tubes, galvanometers and direct electrical stimulation of muscle, and the explanations Duchamp gave of the manner of operation were not fully enabling: but his claim was that “it was well connected, it could almost work”. It is not clear that the technology of the time was capable of remote transmission of desire, or of other forms of love, except indirectly through coded transmission of language, but electrical stimulation of muscle (frogs' legs) through radio transmission was established and it is clear that this work is key prior art in the area of remote actuation of desire.

Duchamp specifies (according to Henderson) that a “bride” broadcasts a wave signal for the transmission of desire generated by excitation of a “desire magneto” indirectly connected to her “sex cylinder” and transmitted through an antenna of a form then known in the art that Duchamp refers to as a “pendu femelle” to a plurality of bachelors. The bachelors receive the signal through antennae described as “sieves” connected to a type of resonator and magneto-mechanical detector that Duchamp refers to as a “chocolate grinder”; and the Bachelors themselves are “Malic molds”—types of Crookes tubes filled with “inert illuminating gas” that are excited by the received signal.

The proposed system is reciprocal, (Henderson): “The other emitter of waves or signals in the Glass is the Juggler of the Centre of Gravity or Handler of Gravity, whose “ball in black metal” projects “waves of disequilibriun” towards the Bride.”

This Juggler in turn is driven by the Boxing Match, which contains both First and Second Rams and is operable to launch nine shots to the realm of the Bride. The bride is said to be ionized, or “stripped bare by the bachelors” in this process.

Duchamp's proposed apparatus, while not apparently functional, is better adapted to the art of love than is anything in the more recent practical art, in that it explicitly recognises the complex interactions involved in love and desire.

### The Feedback Problem

Love cannot reliably be found by any “open-loop” method such as implemented in Yenta or Lovegety (see below), because in the majority of cases it develops over a period of courtship in which plural participants interact in such a way as to release information to one another in a controlled fashion, modifying their own behaviour and desires in response to each step. Most prior art, apart from Duchamp's, suffers from this weakness in its underlying model.

Duchamp's implementation shows a complex two-way interaction between a bride and plural bachelors involving excitation by a desire magneto leading to ionization (“stripping bare”), communication involving splendid vibrations, and cinematic blossoming powered by release of energy contained in love gasoline. It also makes explicit the social context, with the bride interacting with girlfriends as well as with the Bachelors.

### The Deceit Problem

The prevalence of deceit in the pursuit of love is well understood in art (Donne) and is a major drawback with all known methods and apparati. Deceptive behaviours occur because progress to courting behaviour may be valuable for reasons other than the pursuit of love, such as short-term sexual gratification, power, status, revenge, winning a bet, or access to jewelry, art, income streams or other valuable property. Even if no property and little time are lost, they cause “false positives” early in courtship, which waste emotional investment.

Self-deception is also well-known in the art of love. Like deception of others, it can arise from motivations to power or status, and it can also be encouraged by mass media such as the entertainment industry and churches “selling” particular ideals for their own purposes.

Overt negotiation, such as in arranged marriages or Internet dating service, explicitly describes the actual characteristics of the love-seeking party and the sought characteristics of the desired. Overt mechanisms are often ineffective because people have very poor conscious understanding of their desires (self-deception) and because they encourage deception: a dishonest or petty seducer may claim to love art rather than admitting to his preference for bearbaiting.

Subvert negotiation involves visible tokens (signifiers) (e.g. type of car) together with conventions that relate this signifier to a collection of other possessions, attributes or behaviours. Response to the signifier is unconsciously induced by response to said collection of possessions, attributes or behaviours. Cultural mechanisms describe these relations and create the convention. Subvert mechanisms succeed better than overt ones in limiting deceptive behaviour, because rational processes are less applicable, however the conventions are often well-enough recognized that they become partially overt: the cad may observe an attractive woman in Doc Martins and choose to mention Lukacs rather than Norman Rockwell.

This invention is concerned with initial acquisition and development of candidate partners with a high probability of successful courtship. It automates subvert matching over large pools of candidates with excellent protection against deception, and can monitor and manipulate a complex interaction over an extended time.

### Overt/Covert Art

The “lovegety” and “flirtgety” devices described in [refs here] are simplified implementations of the Large Glass, using simple modern radio transceivers to replace the desire magnetos and related apparatus, using commercial dry cells to implement the reservoir of love gasoline, and with much less ambitious transducers of desire. They automate an approach similar to that of a lonely hearts advertisement: the user describes his/her sex by purchasing an appropriately coloured device, then sets switches to describe behavioural preferences from a narrow menu of options (“karaoke” or “walk in the woods”). The target audience is bearers of similar devices within a limited distance, so these devices must be used in social situations such as parties, grocery stores or church meetings where suitable other bearers are likely to be present.

One difficulty with this method is that the menu of choices is quite limited—often offering no representation for homosexual preferences, for example.

Yet another difficulty is the danger of deception, as for any overt technique, as when men simulate sensitivity.

Another difficulty is one inherent in all overt methods: people rarely know what they want, or they deceive themselves. The flaw here is that courting is seen as a form of shopping, in which the wise shopper works from a list, and conscious mediation in producing the list increases the motivation to self-deception. There is a body of philosophy that holds that self deceit can be neutralized by the program “know thyself”, although no embodiment of this program is known. Its ultimate goal, if feasible, is purification or elimination of desire: this would reduce the market for our device. We do not believe that this is likely to be a serious problem, given the antiquity of the program and the paucity of results.

Yet another difficulty is the limited range of the device, which may require a manufactured social situation.

There is also the danger of deception by electronic interception (“sigint” or signals intelligence): a seducer may progressively scan possible settings merely in order to identify the desires of an attractive quarry—then claim to love long walks in the woods rather than admitting to a preference for monster truck rallies.

Lovegety attempts to implement a covert negotiation (the negotiation of match occurs between two mechanisms without the users being notified of the results unless they are successful) but with overt description of desire (“karaoke” etc.).

“Yenta” (Foner, op. cit.) implements another covert negotiation with overt objectives, in which users covertly identify others to whom they are attracted, and then pairs are notified only of mutual attraction. This assumes explicit knowledge of the user's own desires and that the user knows enough about other users to form a judgement, and its main advantage over a straightforward proposition is the avoidance of embarrassment. Yenta is essentially a technical implementation of negotiation through a trusted intermediary as described in (Shakespeare, Chaucer and Ovid) and has the same advantages and disadvantages.

The use of trusted intermediaries is weak because of concerns for: security, since the database may be altered or read improperly, causing embarrassment; commitment, since notification by the system is an admission of attraction, and difficult to deny later when contact is found undesirable; grief and insult, since lack of notification after identifying another implies that the desired does not reciprocate; deception in the form of trifling, since a malicious user may merely identify a large number of others in order to learn about their interest without reciprocating in good faith.

The use of single-step negotiation is a key weakness, since it fails to recognize feedback mechanisms: Yenta only implements a single level of flirtation in what is known in the art to be a complex process of many stages and alternative paths (Capellanus). The “commitment” and “grief” failures are due to this weakness.

A brothel is prior art for the process of explicit identification of a preferred partner, and directly maps the well-established model of shopping to the gratification of simple sexual desires. Yenta is an implementation of this method with the addition of a requirement for symmetric desire and a simple method for partially suppressing knowledge of unreciprocated desire. Lovegety is a further abstraction in that it permits description of the partner in general terms rather than explicit identification, but shares the fundamental weaknesses of any method derived from the underlying shopping model. SUBVERT ART

By choosing to drive a particular type of car, a male may display naive vigour (Camaro), wealth (Lamborghini), moderation (Volvo), adventurousness (Land Rover), etc. These general characteristics are then associated with clusters of behaviours: a Camaro owner may be expected to live with his parents, to drink Jack Daniels, and to want rough sex; a Lamborghini owner can be expected to drink expensive wine, own a yacht and a beach house, travel to exotic locations, and give jewellery to mistresses; a Volvo owner may be expected to have a solid professorial job, at most one divorce, and a large and boringly tasteful house in a quiet uptown neighbourhood; and a Land Rover owner can be expected to have a cottage and a collection of travel books and to enjoy whitewater rafting. Similarly a female may drive a Beetle (“free spirit”), a Harley (“zipless sex”) or a '68 Mustang (“tomboy romantic”). The car is a display of preferences, like the settings of a Lovegety or FlirtGety, in a conventional form, and is used as a courting signal.

The conventions themselves may be determined in a weak fashion by the relation between inherent properties of the products (maximum speed and collision safety record, for example) and desired characteristics (aggression or security, respectively), but are now largely defined explicitly by the marketing organizations that sell them. Companies with marketing organizations able to bring particular brands into the “dictionary of desire” this way are rewarded by being able to sell the branded products at very high margins.

The key advantage of conventional display over overt (“shopping-list”) methods is that it is possible for a participant to buy a car or admire the owner of a car “because it's cool” or “because it's safe” or “because it's practical” without having to explicitly admit to him/herself what the implied preferences are. This makes self-deception harmless, because the internal mechanisms of sexual or romantic desire can communicate directly (through desire for a product) without conscious mediation. Unfortunately these conventions publish fairly quickly, as satirists and artists of various types detect and track them and bring them to public attention, so that participants become self-conscious of the meaning of their choice of a car (or clothing style, preferred liquor, or public entertainment); once explicit, lifestyle degenerates to overt selection. Since the need for subvert negotiation remains, branding is a ongoing campaign against art. DATA MINING

“Data mining” is an emerging technology in which large databases are searched for patterns of behaviour (in particular of consumption) with commercial value. Typically the semantics/meaning of the pattern is regarded as irrelevant. In a typical example, large databases of grocery purchases are searched to find groups of products often bought together and this information is used to decide which coupons to offer to a particular shopper. In broad terms this technology is naturally subvert, and hence a natural candidate for use in romance, but has not yet been applied in a useful way.

### Poetry and Art

The subtlety of language permits courtship, in particular when it is used in forms (such as poetry) in which it is conventional to use multiple meanings, sounds and connotations of words and phrases to communicate in an ambiguous manner. This permits the use of deceit to subvert conscious interference. Also, since language is conventionally interactive, it permits feedback; and since it can be published or declaimed it permits broadcast of desire to enhance initial contact (cf. Byron).

Other arts, including but not limited to music, sculpture and painting, can be and frequently are used in the same way.

The arts, however, require a certain level of skill and do not necessarily involve the use of consumer products. These requirements for skill, sensitivity, knowledge or intelligence limit the available market, and lack of a revenue model makes them worthless to pursue.

### Desired Invention

An apparatus or method is desired that enables its owners or subscribers to be led into relationships that will, for a time, diminish their loneliness. This apparatus or method must be subvert (in the sense of hiding knowledge of the underlying patterns of desire from all participants), must have the flexibility to respond to new types of desire as they are manufactured by marketing and the cultural industries, must be compatible with iterative or feedback methods of kindling love and desire, and must be commercially viable.

For optimum commercial value, the method or apparatus must be protected by intellectual property law, and must be as ubiquitous as air, so that there is no practical method to find love without it. Duchamp's Green Box explains:

Establish a society in which the individual has to pay for the air he breathes (air meters; imprisonment and rarefied air, in case of non-payment simple asphyxiation if necessary (cut off the air)

### SUMMARY OF THE INVENTION

The present invention is a novel method and apparatus for finding love. More specifically, the apparatus and method are operable to induce the likelihood of a match by means not under the direct control of the subjects, particularly means that detect patterns of behaviour inductively and modify them. These means may be designed explicitly to detect certain characteristics, or may be designed more abstractly to recognize patterns without any known interpretation.

### BRIEF DESCRIPTION OF THE DRAWINGS

Preferred embodiments of the present invention will now be described, by way of example only, with reference to the attached Figures, wherein:

FIG. 1 shows a plurality of persons carrying embodiments of the device, with radio communications illustrated among them;

FIG. 2 shows a person wearing a plurality of embodiments of the device, all connected by wireless or wired means;

FIG. 3 shows a schematic representation of the components of the device in FIG. 1;

FIG. 4 shows a schematic representation of a distributed implementation of the device in which groups of components or entire devices communicate with each other to act as a more capable device;

FIG. 5 shows a plurality of persons judging the merits of a candidate and explicitly programming his or her device;

FIG. 6 shows a software object stack representation of an exemplary implementation as a collection of intercommunicating managers for software applets, hardware resources, and a modeller of the subject's pleasure, including sensors and estimators of metrics.

FIG. 7 shows a component representation of the resource manager component of the active database system contained in the memory illustrated in FIG. 2;

FIG. 8 shows a component representation of the applet manager component of the active database system contained in the memory illustrated in FIG. 2;

FIG. 9 shows two component algorithms of a simple applet operable to warn the user that another user with a more impressive history of lingering is in radio range.

FIG. 10 shows an embodiment of the device in an earring, with sensor operable to detect heartrate and actuator operable to whisper messages; together with an embodiment of the device in a nose stud, with sensor operable to detect breathing rate and actuator operable to dispense pheromones in order to subliminally excite the subject;

FIG. 11 shows an embodiment of the device in a chest belt, with sensors operable to detect heartrate and breathing rate and depth, and actuator operable to constrict chest;

FIG. 12 shows an embodiment of the device in a nipple clip, with sensors operable to detect changes in state of erectile tissue and actuator operable to apply mild electrical shocks;

FIG. 13 shows embodiments of the device partially embedded in a subject, with sensor operable to detect odours or sound and actuators operable to stimulate a single neuron or to inject artificial hormones or psychoactive chemicals;

FIG. 14 shows a representation of the contents of a graph database representing the sexual activities of the members of a suburban swingers club;

FIG. 15 shows a representation of the contents of a graph database representing the sexual activities of the regulars at a public washroom;

FIG. 16 shows a representation of the contents of a graph database representing the confessional activities of a small congregation.

### DETAILED DESCRIPTION OF PREFERRED EMBODIMENTS OF THE INVENTION

Subjects wear devices equipped with computing, communication, sensors and actuators. A typical device senses the proximity of others by means of its communication system, and exchanges simple “applets” and data objects with them. An operating system in the devices executes certain applets according to a rule that gives greater computing resources to applets whose decisions have been found successful in the past, including giving successful applets access to actuators so that they can attempt to modify the behaviour of their subjects. A device would generally have an initial collection of applets known to be broadly successful, but new applets could be introduced to the community at any time and would have the power to reproduce if successful.

The success of the system depends on ensuring that applets with desirable behaviour survive and propagate. Their propagation from one device to another is essentially viral, with the individual devices modelling the state of mind of their bearers and favouring the infections that can most cheaply predict their desires; and by allowing the viruses to touch their bodies through a variety of actuators—sounds, smells, electrical shock, micro-injections—devices arrange that these viruses of desire will succeed best if they can manipulate estimated desire to make their predictions more accurate.

Two particular types of data object are envisioned as useful in an initial embodiment: a table of (name, value) pairs and a labelled graph of interactions. It is envisioned that data objects generally permit any applet to read them and to add new entries, but that overwriting of existing entries is controlled through a cryptographic signature mechanism. Objects should in general also implement specialized garbage collection methods that allow the operating system to save storage by deleting data saved by unsuccessful applets.

An interface for the (name, value) pairs, for example, can have a single instance of a Table, with methods including:

Table.Set(Name, Value, Signature)

Value=Table.Get(Name)

size=Table.SpaceOf(AppletID)

void Table.Delete(Name)

An applet “linger” can use this mechanism to estimate the attractiveness of the owner of a device, for example, by estimating how long other owners usually stay nearby (polling the communications interface to determine this at the current time, and averaging the result with an earlier estimates by some formula such as

“LingerTime=0.99*LingerTime+0.01*LatestTime”), then setting

Table.Set(“Linger”, LingerTime, LingerSignature)

This applet could then compare the LingerTime values for two subjects as they approached each other, perhaps before they have had an opportunity to see each other, and make recommendations using an earpiece transducer; for example telling a moderately attractive male to suck in his gut on the approach of an attractive female.

This applet uses a small amount of memory resource (the LingerTimetable entry), which is therefore not likely to be tidied away, and occasionally requests the use of the relatively expensive earpiece resource. If its advice is found to be good, then the operating system will allocate it a higher priority, and its access to the earpiece will become more probable.

Note that the subject does not explicitly set LingerTime, and therefore cannot overtly claim to be more attractive (or “lingery”) than he or she actually is. In general the author of an applet will not explain or publicize its algorithms lest users then modify their behaviour to mislead the algorithm: for example sleeping with a collection of devices so as to appear to cause lingering.

The operating system uses a nonlinear optimization algorithm and a defined criterion to select how many resources to give each applet. An example of such a criterion is that an applet that correctly predicts that a particular subject will spend the night with the owner of the given device would score highly, and in general accurate prediction of duration of contacts would score positively. An example of a suitable optimization algorithm is one in which resources are initially allocated equally, and then the resources available to each applet are increased by a small percentage for each applet scoring a more accurate estimate than the median and decreased slightly for each applet whose estimates are less accurate than the median.

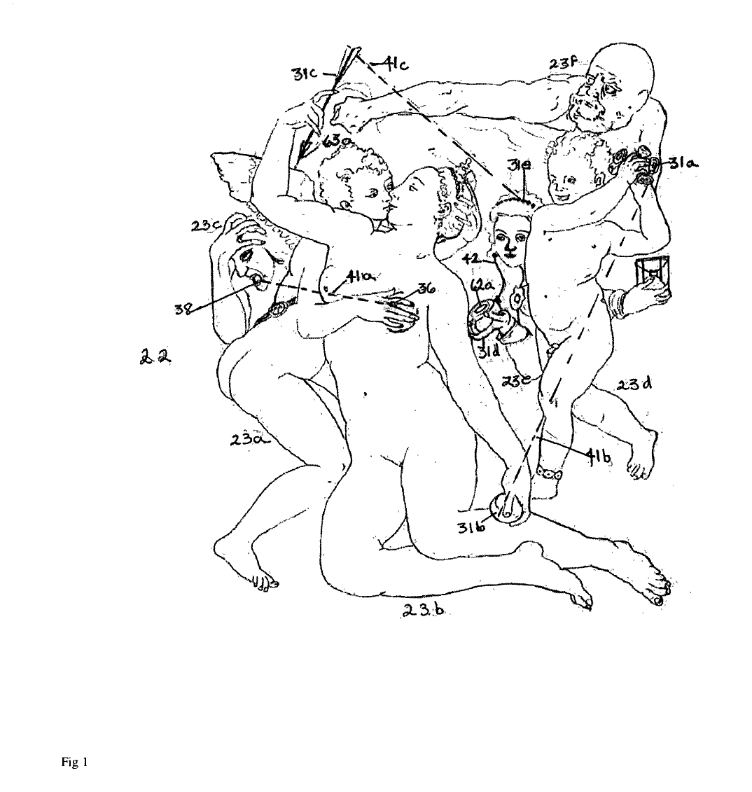

According to FIG. 1 a community 22 of subjects 23, such as concertgoers, wearing instances of device 31, such as will be described below, are led to interact with others likely to be objects of desire by the actions on each said subject 23 of actuators 63, such as those described below, in each device 31 wherein the plural device 31 communicate by wireless links 41, such as by BlueTooth, IEEE802.11b or infrared links or by wired means such as 10-base-T Ethernet.

In the embodiment of FIG. 1 subject 23a Eros caresses subject 23b Aphrodite including direct mechanical activation of a simple pressure switch in nipple device 36, which communicates over wireless link 41a to embedded device 38 in subject 23c Jealousy. The effect is amplified by a further interaction in which wireless link 41b from device 31a held by subject 23d Folly to peach device 31b held by said subject 23b Aphrodite causes it to secrete a sweet-smelling liquid, distracting said subject 23a Eros from the operation of arrow device 31c held by said subject 23b Aphrodite which can be violently but partially embedded in the body of either said subject 23a Eros or said subject 23c Jealousy in order that its actuator 63a may inject an aphrodisiac such as Viagra, hallucinogen such as lysergic acid, muscle relaxant such as curare or other chemical or plural chemicals such as Rohypnol known in the art of seduction. The choice of chemical may be modified by interactions with external parties, such as subject 23e Deceit who carries a handheld device 31d connected by wired link 42 to headband device 31e which is more conveniently placed to make wireless link 41c operable to said arrow device 31c. By means of sensor 62a operable to detect pheremones said handheld device 31d selects which type or combination of chemicals can best be injected to make the behaviour of injected subject 23a Eros or injected subject 23c Jealousy more desirable to both subject 23e Deceit and subject 23b Aphrodite. Other nearby subjects, such as subject 23f Time may have only indirect interactions with the principal emotional drama engulfing subjects 23a through e.

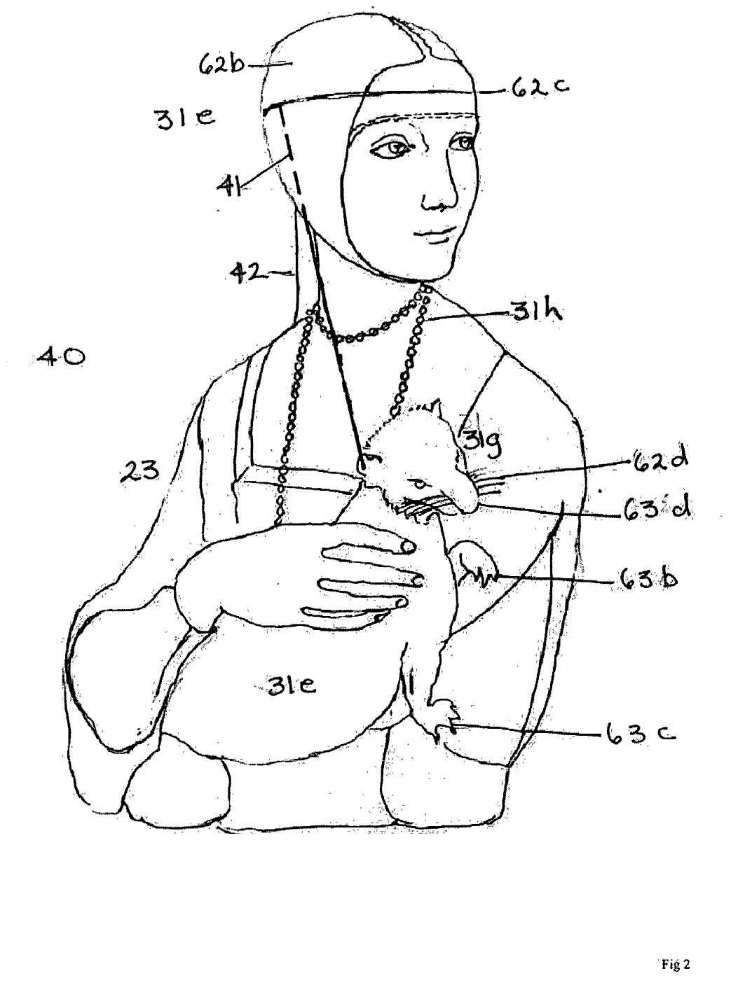

According to FIG. 2 each subject 23 may wear plural device 31, all communicating by wireless links 41 or by means of wired link 42. These plural device 31 combine their actions in such a way as to act towards outsiders as a single more capable distributed device 40 having plural sensors 62 and actuators 63.

In the embodiment of FIG. 2 headband device 31e contains sensor 62b operable to detect local electromagnetic radiation and sensor 62c operable to measure electroencepholographic potentials together with actuator 63b operable to cause a tingling or tightening sensation in the scalp, such as by myoelectrically activating scalp muscles. It is connected to ferret device 31g having whisker sensors 62d operable to detect air currents from movement of nearby bodies together with claw actuators 63b operable to inflict pain or alternately or in combination claw actuators 63c operable to inject venom, aphrodisiacs, pheromones, muscle relaxants, Mickey Finns, sodium pentathol or other chemicals known in the art of seduction. Ferret device 31g may also have tooth actuators 63d operable for the same purposes as claw actuators 63b or actuators 63c, or because of the possibility of scissor action also capable of severing small body parts in a subject 23. Necklace device 31h connected to headband device 31e may simply offer more computing resources, such as memory or battery power.

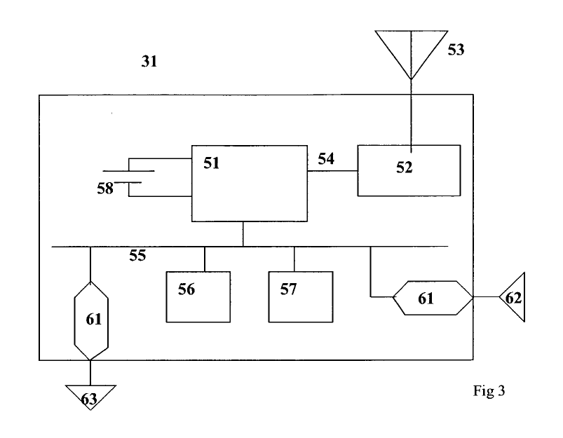

According to FIG. 3 each device 31 contains a CPU 51 such as a StrongArm processor maufactured by Intel, a radio module 52 such as a Bluetooth module manufactured by Conexant, antenna 53 such as a whip, an address and data bus 55 such as that standard with said StrongArm, a flash memory 56 such as a 28F640W18 chip manufactured by Intel, a random access memory 57 such as the IBMN612404GT3B manufactured by IBM, a battery 58 such as a wristwatch battery. It may also contain one or plural actuator 63 or sensor 62, each connected to the address and data bus 55 by means of a signal conditioning circuit 61.

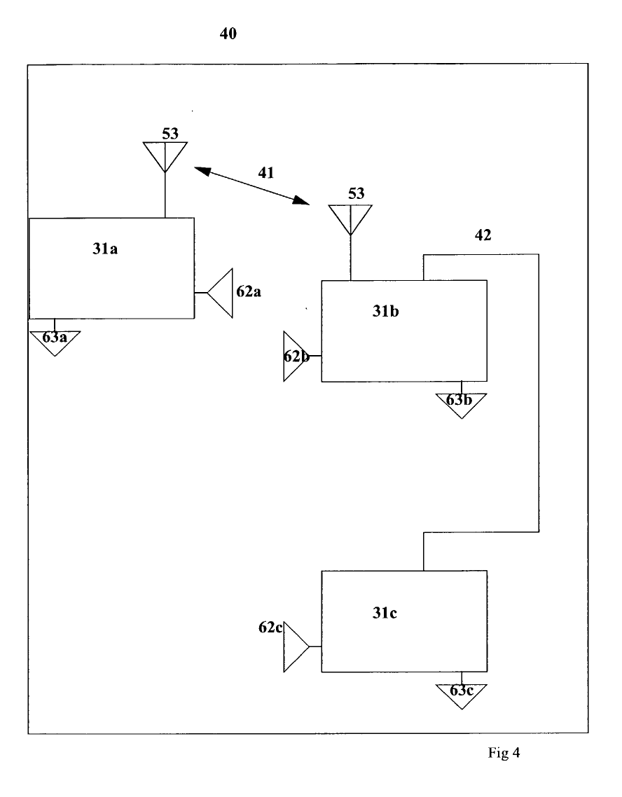

According to FIG. 4 plural device 31 may be connected by means of wireless links 41 or wired links 42 to form a more capable distributed device 40.

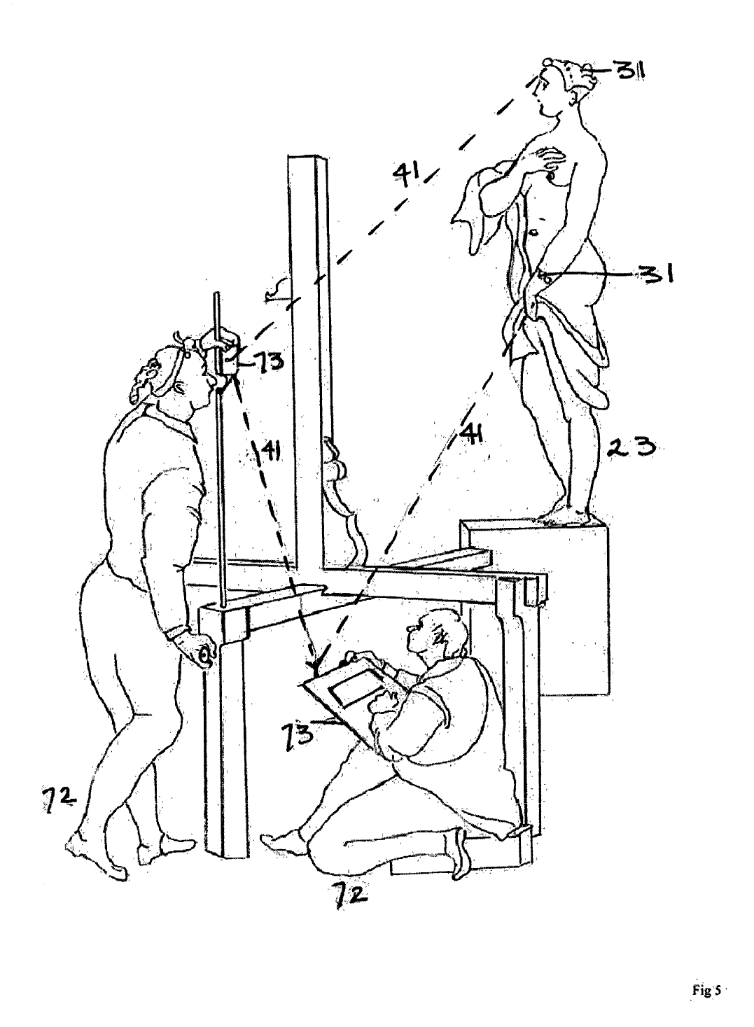

According to FIG. 5 a device 31 or plural devices 31 may be programmed by a board 71 of judges 72, such as retired medical professors, who evaluate the subject 23 intended to bear the said devices 31, in terms of characteristics deemed to be likely to substantially affect his or her attractiveness to various groups, by means of programming devices 73 such as laptop computers. The programming devices 73 and love device 31 may all communicate by means of wireless links 41. It will be apparent to one versed in the art that this communication may also take place by wired means.

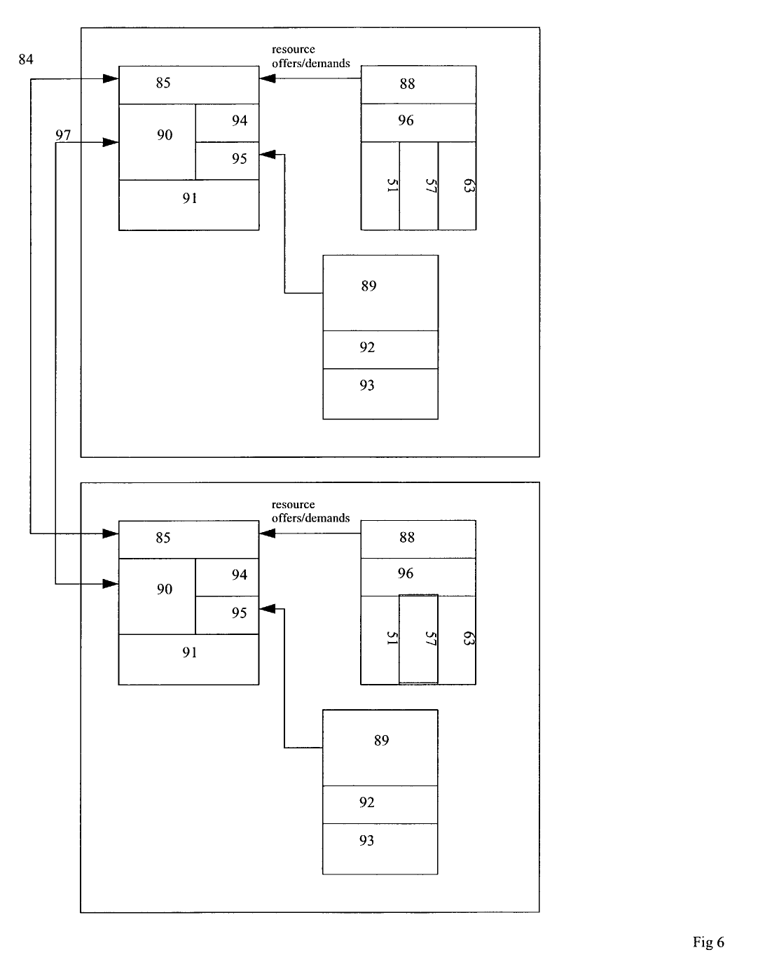

According to FIG. 6 the software running in a device 31 may contain an applet management system 85 which includes an applet database 90, a score keeper 94 that tracks the accuracy of predictions registered by applets in a prediction registry 95 and gives applets access to a collection of standard objects including labelled graph and table 91. The applet management system 85 is operable to exchange applets with other instances of a device 31 and to give applets greater or lesser proportions of resources offered by resource allocation manager 88 as a function of their cost/performance ratio as calculated from their scores and their resource usage; this algorithm being chosen to maximize likelihood of correct predictions from the subject desire and pleasure modellers 89 with minimum use of resources.

The software running in device 31 may also contain a resource allocation manager 88 which includes software operable as a usage monitor and cop 96 that estimates and controls usage of such resources as CPU 51, random access memory 57 and sensors 62 with algorithms well known in the operating system art. This resource allocation manager 88 may for example operate by having targets for average utilization of each resource and offering increases or demanding reductions in such use from the applet management system 85 according to a message interface of a type well known in the art of object-oriented programming systems.

The software running in device 31 may also contain a subject desire and pleasure modeller 89 which uses estimators of standard metrics 92, which process raw data from sensor drivers 93 to measure such things as heart rate and pupil dilation.

It will be apparent to one skilled in art that there are many metrics either for pleasure or desire. At a commercial level it may be expected that devices 31 implementing metrics that correspond to popular concepts of pleasure will be preferentially selected by the market, according to the dictum “I may not not much about love, but I know what I want”. In this case, however, the covert nature of the algorithms means that subjects 23 will not be aware of how they are being manipulated; this would, for example, allow for commercial success of a device 31 so designed as to make mood swings extreme while taking credit for the positive swings and hiding its participation in the negative swings. From an abstract point of view this is simply a definition of the criterion “pleasure” and for the purpose of this disclosure the term shall be taken to comprise all manipulations of mood that tend to enhance dependence on devices 31.

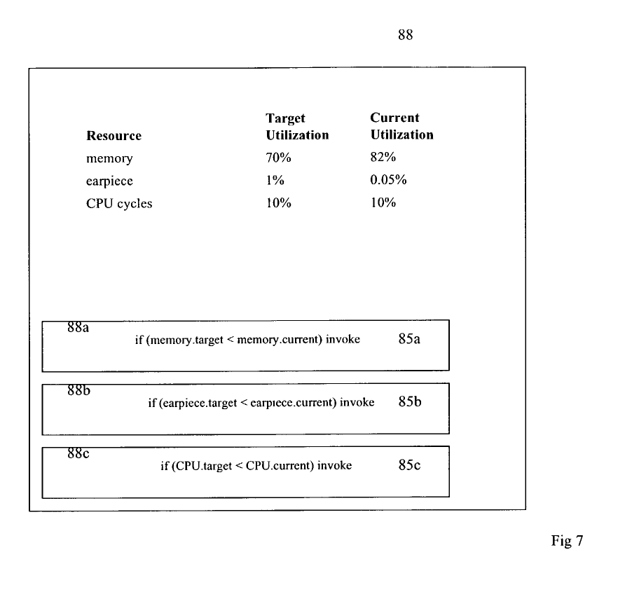

According to FIG. 7 the random access memory 57 of each of the devices 31 described above contains a resource allocation manager 88 operable to increase or decrease the resources available to plural Java applets 84 depending on the success of their predictions. It is composed of specialist resource allocation managers 88a through c each operable to drive usage of a particular resource towards a target by passing requests on to the applet manager described below.

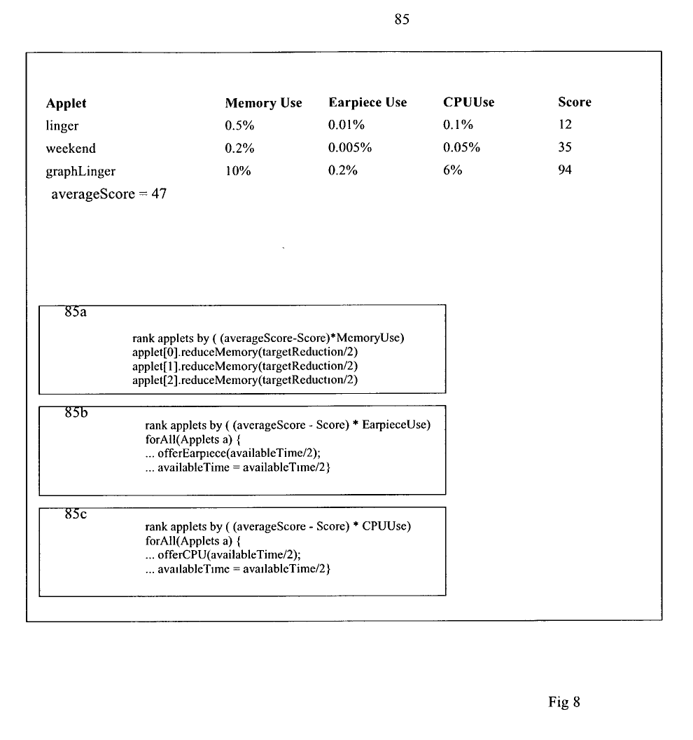

FIG. 8 shows an applet management system 85 operable to distribute resources among plural Java applets 84 according to their consumption and success at prediction. It contains specialized component applet management systems 85a through c with algorithms that rank applets and distribute resources or cuts in resources as required by the resource allocation manager 88. It would implement other methods, including ones that download applets from new acquaintances so that successful ones can propagate. It would tend to prefer to download applets with high scores and low resource usage.

It will be apparent to one skilled in the art that many algorithms, broadly called evolutionary algorithms, exist that have the general properties required for an applet management system 85, and that these will have different success in propagating desired viruses. At a commercial level, devices 31 with good behaviour will be selected by the market.

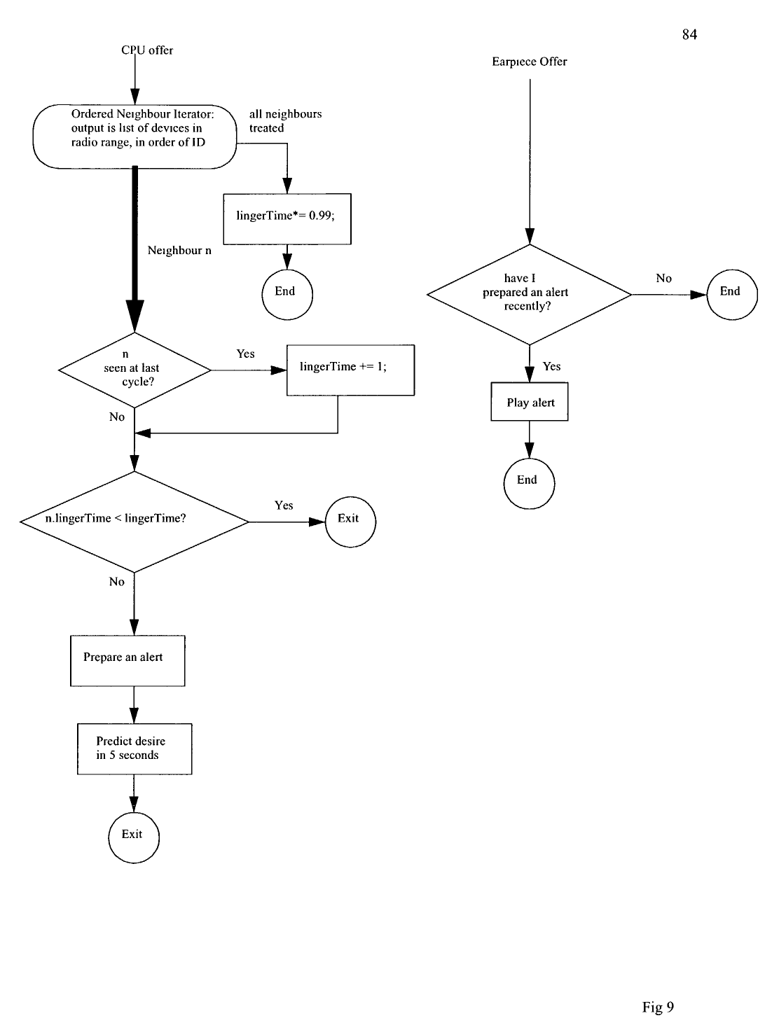

According to FIG. 9 an exemplary Java applet 84 “linger” would contain methods responsive to offers of CPU and earpiece resources from applet management system 85. The CPU offer would be exploited by checking for neighbouring devices with “lingerTime” greater than that of the current device. The earpiece offer would be exploited by playing a prepared message such as “sweet!” if a recent high-lin-gerTime neighbour had been found, and in this same case would predict a momentary surge of desire within the next ten seconds. If this algorithm thus succeeds in alerting the subject 23 to the presence of an attractive person within radio range, then for example a male subject 23 may suck in his gut and look around, perhaps seeing an attractive woman and fulfilling the prediction of desire. This in turn will improve the score of this applet, and hence its probability of survival.

It will be apparent to one skilled in the art that many similar algorithms can be developed, each having several or all of the characteristics that it attempts to predict desire, that it may use access to actuators 63 to improve the likelihood of its prediction being accurate, that it uses sensor data, and that it communicates with applets or standard objects in nearby devices 31 to improve its accuracy. These applets will be written to be parsimonious in their usage of resources in order to enhance their chances of survival in an individual and propagation to contacts, thus using viral propagation to the advantage of the system. These applets may be developed by anyone competent to develop Java code, thus enabling the development of a large supply of viruses.

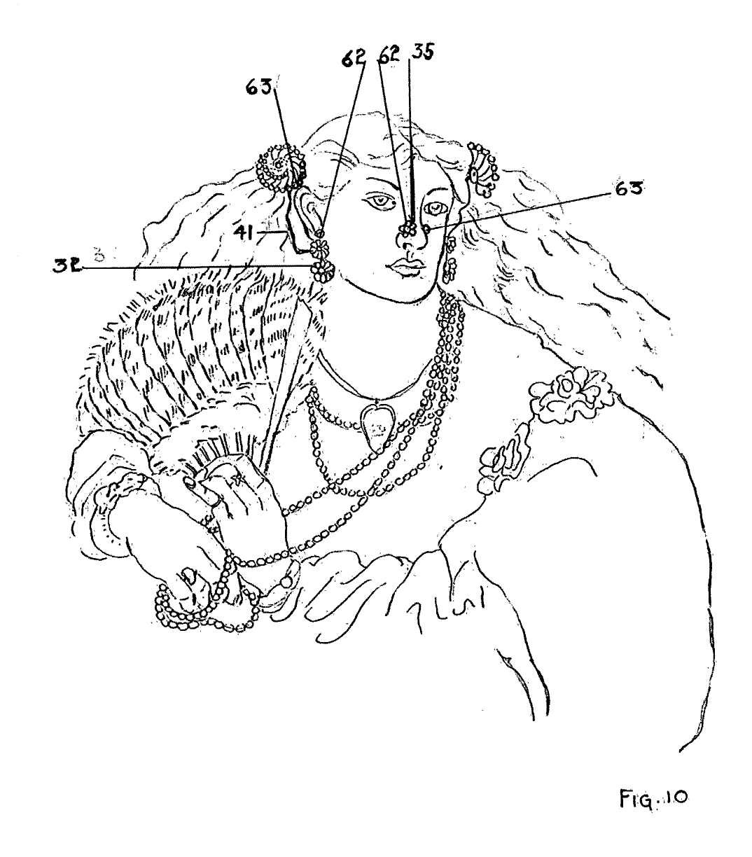

According to FIG. 10 an earring device 32, which is a particular type of device 31, may be constructed as shown in FIG. 3 sup wherein the sensor 62 is operable to detect heartrate and the actuator 63 is operable to make a variety of sounds such as whispered messages or songs. Also according to FIG. 10 a nose stud device 35, which is a particular type of device 31, may be constructed as shown in FIG. 3 sup wherein the sensor 62 is operable to detect pheromones and carbon dioxide and the actuator 63 is operable to dispense a variety of pheromones and scents

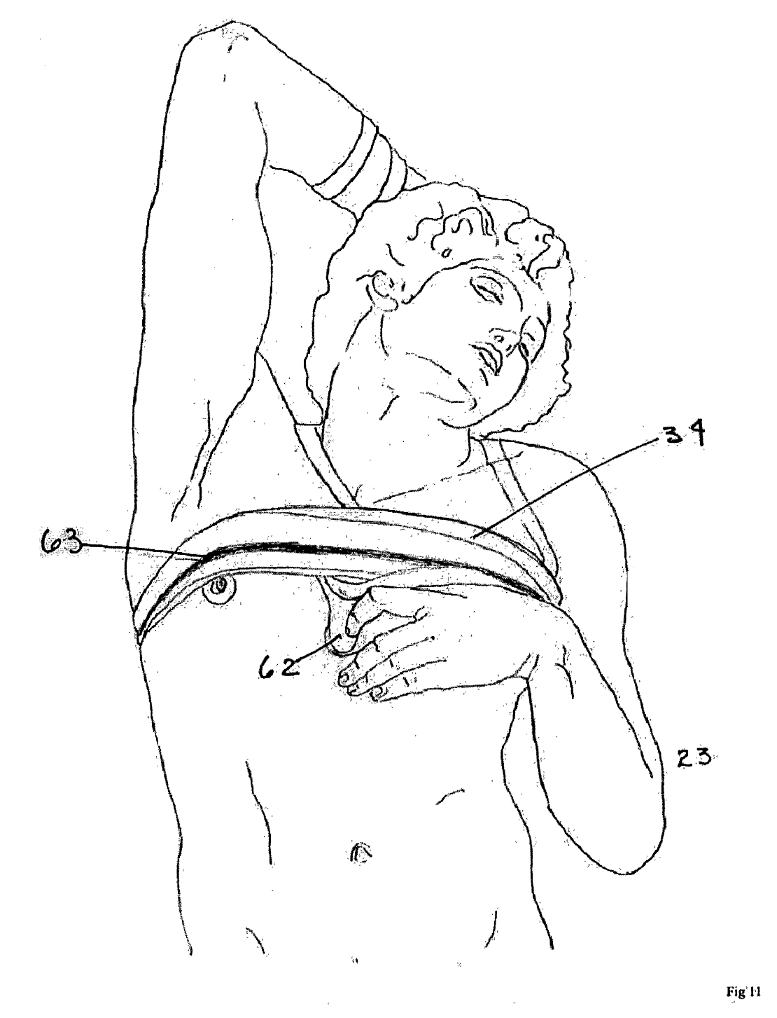

According to FIG. 11 a breast device 34, which is a particular type of device 31, may be constructed as shown in FIG. 3 sup wherein the sensor 62 is operable to detect heartrate and breathing rate and depth, and the actuator 63 is operable to constrict the chest in patterns that simulate rapid breathing or that temporarily restrict breathing altogether.

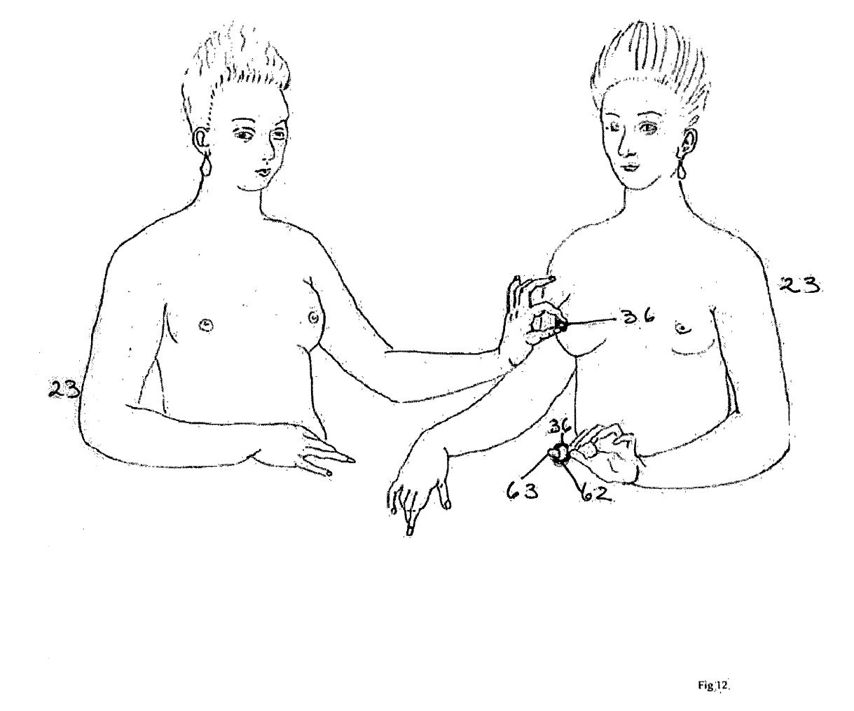

According to FIG. 12 a nipple device 36, which is a particular type of device 31, may be constructed as shown in FIG. 3 sup wherein the sensor 62 is operable to detect changes in state of erectile tissue and the actuator 63 is operable to apply mild electrical shocks.

It will be apparent to one skilled in the art that a similar device could be developed to detect tumescence in the penis and to stimulate it with shocks, warmth, vibration or topical injections of nitric oxide.

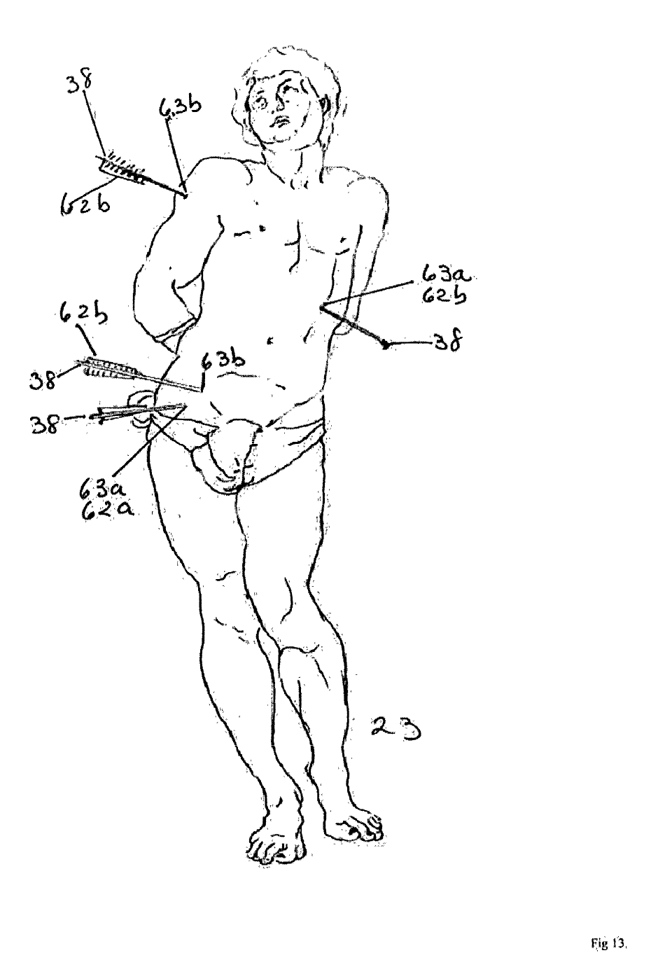

According to FIG. 13 an embedded device 38, which is a particular type of device 31, may be constructed as shown in FIG. 3 sup wherein the sensor 62a is operable to detect activity of a single neuron and the actuator 63a is operable to stimulate a single neuron. Alternative interfaces for an object of this type could include a sensor 62b operable to detect sounds characteristic of opera or Gregorian chant and an actuator 63b operable to dispense psychoactive chemicals or hormones.

A second important data object is the labelled graph, which is capable of representing complex behaviours without direct reference to their semantics, such as sexual preference. In the preferred embodiment a graph that represents a subset of the transitive closure of those subjects with which the given subject comes into contact is labelled with (name, value) pairs, such as the LingerTime data described above. The data now refers to Lingering with specific individuals, rather than with people in general, so that more subtle types of attraction may be represented.

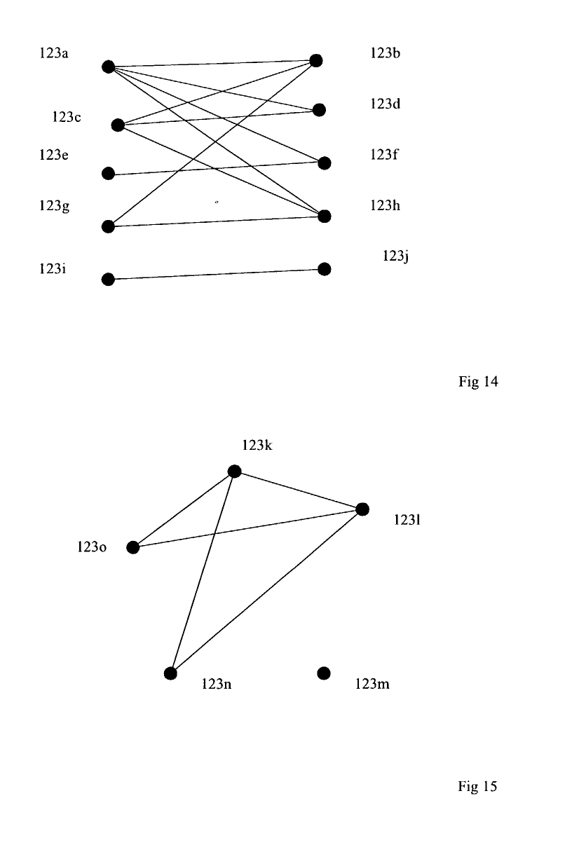

According to FIG. 14 a portion of labelled graph 83 may represent the sexual activities of the members represented by nodes 123a through j of a suburban swingers club, where only edges labelled with data suggestive of sexual activity are shown. Note that there is extensive contact, but that the nodes (representing subjects) may be divided into two distinct groups with contact occurring from one group to another but not within the groups. This bipartite graph is characteristic of promiscuous heterosexuality. An applet able to recognize this pattern would be able to introduce members of the Dallas and Orange County swingers clubs to each other even with no common contacts, because on the first encounter between members of the two groups (at a Jester's conference, for example) the applet would recognize that both subjects shared this activity.

This graph is semantically ambiguous, in that it could equally represent the violent physical contacts among members of two five-a-side basketball teams. Note that it would still function to introduce basketball players to each other. In the case where the sensor device or recording applet is unable to distinguish between sexual and game contacts it would also tend to introduce basketball players to suburban swingers: depending on the reactions of the parties so introduced the given applet would be more or less apt to survive. This is an example of applet behaviour that is likely not to have been expected by the original applet author, and hence of the importance of the management structure herein disclosed.

According to FIG. 15a portion of labelled graph 83 may represent the sexual activities of the regulars represented by 123k through n at a particular public washroom. Note the presence of a highly connected subgraph, which could suggest homosexual or pansexual behaviours within the group. Note also that the individual represented by 123m does not participate. Again, an applet able to observe the existence of this pattern in the database of a newly acquired neighbour would be able to suggest a potential relationship; and if that relationship were successful it would then be able to strongly recommend members of the new partner's group.

Again the semantics of the graph of FIG. 15 are ambiguous. If the sensor or applet reporting this structure is unable to distinguish between sexual activity and the exertion and proximity at a gymnasium or religious revival meeting, it will introduce workout enthusiasts to each other and also to promiscuous homosexuals and fundamentalist Christians.

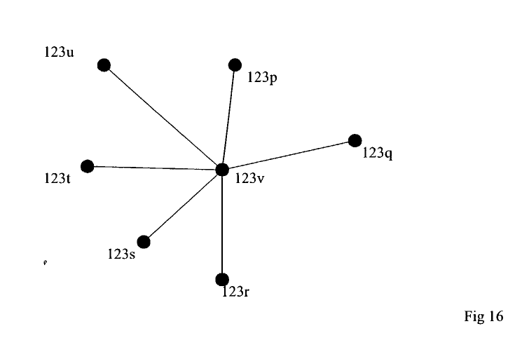

According to FIG. 16 a portion of labelled graph 83 may represent the activities of a small congregation 123p through u confessing privately to a minister 123v.

Equivalently the graph could represent the sexual activity of a prostitute represented by 123v: again a large number of nodes all connect to a single “central” one but not directly to one another. This would be ambiguous if the only sensor were “proximity” as judged by the presence and duration of radio contact between the devices of the various subjects. A more discriminating sensor would be required make it possible to distinguish the shepherd from the black sheep.

Various alternatives and enhancements to the embodiments described above will be apparent to those skilled in the art and are not intended to be excluded from the scope of the present invention which is defined solely by the claims appended hereto.

What is claimed is:

1. A device operable to aid in finding love comprising:
wireless means of communication;
computing means;
portable power source; and
software conforming to a convention of communication between the given device and similar devices.

2. The system of claim 1 further including an actuator that directly stimulates a sexual response in the bearer.

3. The system of claim 2 in which the actuator causes pain.

4. The system of claim 1 with a sensor operable to predict desire.

5. The system of claim 1 further including means to predict desirability.

6. The system of claim 1 in which the said software is operable to propagate, to other devices, software components that predict desire, in a viral manner.

7. The system of claim 6 in which the software tends to preferentially propagate viruses that use few resources.

8. The system of claim 7 which contains a software object operable as a resource manager.

9. The system of claim 8 in which the resource manager has target settings for resource utilization.

10. A collection of devices operable to manipulate human moods and sensations in such a way as to create a psychological or physical dependence on them.

11. The system of claim 10 wherein the devices interoperate to exchange software objects.

12. The system of claim 11 in which the objects exchanged are Java applets.

13. The system of claim 10 in which the objects are chosen for their ability to stimulate sexual desire.

14. The system of claim 10 in which the objects are chosen for their ability to stimulate spiritual ecstasy.

15. A system comprising a method and apparatus manipulative of human desire in such a way as to create a new and monstrous hybrid being whose desires and formerly human interactions have been shaped for commercial benefit.

16. The system of claim 15 in which the apparatus is beautiful.

17. The system of claim 16 in which the apparatus is a living thing.

18. A system to manipulate human desire in such a way as to create a new and monstrous hybrid being whose desires and formerly human interactions have been shaped for ideological benefit.

19. The system of claim 18 in which the apparatus has an apparent function different from its intended function, thus hiding it at the level of intention.

20. The system of claim 19 in which the apparent function is to enable the pursuit of happiness.
